(19) United States
(12) Patent Application Publication
(10) Pub. No.: US 2025/0261873 A1
(43) Pub. Date: Aug. 21, 2025

(54) ELECTRODE FOR AUTOMATIC SKIN ABRASION AND IMPEDANCE MONITORING

(71) Applicant: RHYTHMLINK INTERNATIONAL, LLC, COLUMBIA, SC (US)

(72) Inventors: Brian Petree, West Columbia, SC (US); Michael L. Brown, Columbia, SC (US)

(21) Appl. No.: 19/055,658

(22) Filed: Feb. 18, 2025

Related U.S. Application Data
(60) Provisional application No. 63/555,530, filed on Feb. 20, 2024.

(51) Int. Cl. A61B 5/0531; A61B 5/259; A61B 17/32

(52) U.S. Cl. CPC A61B 5/0531; A61B 5/259; A61B 17/32; A61B 2017/320004; A61B 2562/0214

(57) ABSTRACT

Various systems for positioning an electrode on a patient are provided. An example system may include an abrasion electrode configured to be placed at a first skin location of a patient. The abrasion electrode includes an abrasion component to abrade the first skin location of the patient and an electrode component to electrically engage with the patient. The system may also include a reference electrode configured to be placed at a second skin location of the patient. The system may further include an impedance determination device. The impedance determination device monitors an impedance between the abrasion electrode and the reference electrode. The system may also include a user indication device. The user indication device causes a notification to be provided to a user in an instance in which the impedance between the abrasion electrode and the reference electrode is within a predetermined range of impedance.

### CROSS REFERENCE TO RELATED APPLICATIONS

This application claims the benefit of and priority to U.S. Provisional Patent Application No. 63/555,530, filed on Feb. 20, 2024, entitled Electrode for Automatic Skin Abrasion and Impedance Monitoring, the contents of which are hereby incorporated in the entirety.

### FIELD

An example embodiment relates generally to electrodes for positioning on a patient's skin, and more particularly, to electrodes that are capable of abrading the skin of the patient during placement.

### BACKGROUND

One of the challenges during an electroencephalogram (EEG) procedure for the technicians is applying the electrodes properly, and ensuring a stable, low impedance connection between the skin and the electrode. Low impedance between the skin and electrode is required to ensure a high signal-to-noise ratio, producing a clean signal representative of brain electrical activity. The standard process for ensuring low skin-to-electrode impedance includes manually abrading the skin using an exfoliating gel or in some cases a blunt needle or other material to remove dead skin cells. Removing dead skin cells is the major purpose of the abrasion as the dead skin cells create a higher impedance barrier.

An EEG technician will measure and mark the electrode locations on the patient's head and then use a Q-tip or other instrument to apply an exfoliating gel to the electrode locations and abrade the skin using the instrument and gel. This process is done for each electrode location on the patient's head which is normally between 10-28 spots, and in some cases much higher. The technician will then place the electrodes on each location using a conductive, sticky paste, and then in most cases apply tape or even a head wrap to ensure the electrodes remain in place. The technician will then perform an impedance check to ensure all of the electrodes are within acceptable impedance range of 10 kΩ (kiloohms), with target ranges of around 5-10 kΩ. However, any electrodes that fail the impedance check must be adjusted or, more likely, removed and reapplied in order to comply with the impedance requirements. As such, there exists a need for a unified system that can provide electrode adjustment and use without additional steps, and without removing the electrode.

### SUMMARY

The following paragraphs present a summary of various embodiments of the present disclosure and are merely examples of potential embodiments. As such, the summary is not meant to limit the subject matter or variations of various embodiments discussed herein.

In some aspects, the techniques described herein relate to a system for positioning an electrode, the system including: an abrasion electrode configured to be placed at a first skin location of a patient, the abrasion electrode includes an abrasion component to abrade the first skin location of the patient and an electrode component to electrically engage with the patient; a reference electrode configured to be placed at a second skin location of the patient; an impedance determination device, wherein the impedance determination device monitors an impedance between the abrasion electrode and the reference electrode; and a user indication device, wherein the user indication device causes a notification to be provided to a user in an instance in which the impedance between the abrasion electrode and the reference electrode is within a predetermined range of impedance.

In some aspects, the techniques described herein relate to a system, further including an abrasion motor connected to the abrasion component of the abrasion electrode, wherein the abrasion motor provides movement of the abrasion component to abrade.

In some aspects, the techniques described herein relate to a system, wherein the predetermined range of impedance is between 5 kiloohms and 10 kiloohms.

In some aspects, the techniques described herein relate to a system, wherein the impedance determination device monitors the impedance between the abrasion electrode and the reference electrode during placement of the abrasion electrode.

In some aspects, the techniques described herein relate to a system, wherein the abrasion electrode and the reference electrode are connected via a lead-wire, wherein the impedance between the abrasion electrode and the reference electrode is monitored via the lead-wire.

In some aspects, the techniques described herein relate to a system, wherein the reference electrode includes a hydrogel pad.

In some aspects, the techniques described herein relate to a system, further including an additional abrasion electrode configured to be placed at a third skin location of the patient, wherein the additional abrasion electrode includes an abrasion component to abrade the third skin location of the patient and an electrode component to electrically engage with the patient, wherein the impedance determination device monitors an impedance between the additional abrasion electrode and the reference electrode.

In some aspects, the techniques described herein relate to a method of manufacturing an impedance determination electrode system, the method including: providing an abrasion electrode configured to be placed at a first skin location of a patient, the abrasion electrode includes an abrasion component to abrade the first skin location of the patient and an electrode component to electrically engage with the patient; providing a reference electrode configured to be placed at a second skin location of the patient; providing an impedance determination device to monitor an impedance between the abrasion electrode and the reference electrode; and providing a user indication device, wherein the user indication device causes a notification to be provided to a user in an instance in which the impedance between the abrasion electrode and the reference electrode is within a predetermined range of impedance.

In some aspects, the techniques described herein relate to a method, further including connecting an abrasion motor to the abrasion component of the abrasion electrode, wherein the abrasion motor provides movement of the abrasion component to abrade.

In some aspects, the techniques described herein relate to a method, wherein the predetermined range of impedance is between 5 kiloohms and 10 kiloohms.

In some aspects, the techniques described herein relate to a method, wherein the impedance determination device monitors the impedance between the abrasion electrode and the reference electrode during placement of the abrasion electrode.

In some aspects, the techniques described herein relate to a method, further including connecting the abrasion electrode and the reference electrode via a lead-wire, wherein the impedance between the abrasion electrode and the reference electrode is monitored via the lead-wire.

In some aspects, the techniques described herein relate to a method, wherein the reference electrode includes a hydrogel pad.

In some aspects, the techniques described herein relate to a method, further including providing an additional abrasion electrode configured to be placed at a third skin location of the patient, wherein the additional abrasion electrode includes an abrasion component to abrade the third skin location of the patient and an electrode component to electrically engage with the patient, wherein the impedance determination device monitors an impedance between the additional abrasion electrode and the reference electrode.

In some aspects, the techniques described herein relate to a method of using an impedance determination electrode system, the method including: positioning an abrasion electrode at a first skin location of a patient, the abrasion electrode includes an abrasion component to abrade the first skin location of the patient and an electrode component to electrically engage with the patient; positioning a reference electrode at a second skin location of the patient; monitoring, via an impedance determination device, an impedance between the abrasion electrode and the reference electrode; and causing a notification to be provided to a user in an instance in which the impedance between the abrasion electrode and the reference electrode is within a predetermined range of impedance.

In some aspects, the techniques described herein relate to a method, further including causing an actuation of an abrasion motor connected to the abrasion component of the abrasion electrode, wherein the abrasion motor provides movement of the abrasion component to abrade.

In some aspects, the techniques described herein relate to a method, wherein the predetermined range of impedance is between 5 kiloohms and 10 kiloohms.

In some aspects, the techniques described herein relate to a method, wherein the impedance between the abrasion electrode and the reference electrode is monitored during placement of the abrasion electrode.

In some aspects, the techniques described herein relate to a method, wherein the reference electrode includes a hydrogel pad.

In some aspects, the techniques described herein relate to a method, further including: positioning an additional abrasion electrode at a third skin location of the patient, wherein the additional abrasion electrode includes an abrasion component to abrade the third skin location of the patient and an electrode component to electrically engage with the patient; and monitoring, via the impedance determination device, an impedance between the additional abrasion electrode and the reference electrode.

### BRIEF DESCRIPTION OF THE DRAWINGS

Many aspects of the present disclosure will be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views. It should be recognized that these implementations and embodiments are merely illustrative of the principles of the present disclosure. Therefore, in the drawings:

FIG. 1 is an illustration showing an impedance determination electrode system positioned on the head of a patient, in accordance with various embodiments;

FIG. 2 is an illustration of an example abrasion electrode and a reference electrode of the impedance determination electrode system, in accordance with various embodiments;

FIG. 3 is a flowchart of an example method of manufacturing an impedance determination electrode system, in accordance with various embodiments; and

FIG. 4 is a flowchart of an example method of using an impedance determination electrode system, in accordance with various embodiments.

### DETAILED DESCRIPTION

The presently disclosed subject matter now will be described more fully hereinafter with reference to the accompanying drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements.

Indeed, many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims.

Throughout this specification and the claims, the terms “comprise,” “comprises”, and “comprising” are used in a non-exclusive sense, except where the context requires otherwise. Likewise, the term “includes” and its grammatical variants are intended to be non-limiting, such that recitation of items in a list is not to the exclusion of other like items that can be substituted or added to the listed items.

### I. Systems and Methods

In some aspects, the techniques described herein relate to a system for positioning an electrode, the system including: an abrasion electrode configured to be placed at a first skin location of a patient, the abrasion electrode includes an abrasion component to abrade the first skin location of the patient and an electrode component to electrically engage with the patient; a reference electrode configured to be placed at a second skin location of the patient; an impedance determination device, wherein the impedance determination device monitors an impedance between the abrasion electrode and the reference electrode; and a user indication device, wherein the user indication device causes a notification to be provided to a user in an instance in which the impedance between the abrasion electrode and the reference electrode is within a predetermined range of impedance.

In some aspects, the techniques described herein relate to a system, further including an abrasion motor connected to the abrasion component of the abrasion electrode, wherein the abrasion motor provides movement of the abrasion component to abrade.

In some aspects, the techniques described herein relate to a system, wherein the predetermined range of impedance is between 5 kΩ (kiloohms) and 10 kΩ (kiloohms).

In some aspects, the techniques described herein relate to a system, wherein the impedance determination device monitors the impedance between the abrasion electrode and the reference electrode during placement of the abrasion electrode.

In some aspects, the techniques described herein relate to a system, wherein the abrasion electrode and the reference electrode are connected via a lead-wire, wherein the impedance between the abrasion electrode and the reference electrode is monitored via the lead-wire.

In some aspects, the techniques described herein relate to a system, wherein the reference electrode includes a hydrogel pad.

In some aspects, the techniques described herein relate to a system, further including an additional abrasion electrode configured to be placed at a third skin location of the patient, wherein the additional abrasion electrode includes an abrasion component to abrade the third skin location of the patient and an electrode component to electrically engage with the patient, wherein the impedance determination device monitors an impedance between the additional abrasion electrode and the reference electrode.

In various embodiments, methods of manufacture and/or methods of use may be provided that provide methods of manufacturing and/or using the system of various embodiments. For example, one method may include assembling of the abrasion electrode, and the reference electrode for use on a patient. In another aspect it may be the method of utilizing the disclosure herein to produce results.

### II. Example Use Case

The outermost layer of the epidermis, the stratum corneum (SC), is composed of dead, keratinized cells and acts as a significant barrier to electrical conductivity. The stratum corneum layer has high resistance (i.e. impedance) and can cause signal attenuation or noise interference in electrode recordings on the head of a patient. For example, the natural impedance of unprepared skin is often above 50 kΩ (kiloohms), depending on factors such as hydration and skin condition. Hydration of the skin creates an ionic path between the electrode and the epidermis below the stratum corneum layer, which allows the transductions of ionic current into electrical current easier, lowering the skin-electrode impedance. Thus, gels may be used to improve electrode readings due to the ionic pathway.

Skin preparation and abrasive techniques remove part of the stratum corneum, allowing better contact between the electrode and the underlying epidermis, where conductive interstitial fluids help carry electrical signals. Types of abrasive methods may include physical and/or chemical abrasion including: application of an electrolyte gel to reduce resistivity of the epidermis, manual abrasion or puncture to reduce the width of the stratum corneum, and application of an abrasive paste or alcohol pad to remove to outermost cells of the skin. However, the use of alcohol to abrade the skin may cause dehydration and increase impedance. As the skin rehydrates itself, the impedance then reduces below its original value.

During the placement of an electrode, a technician has to perform an impedance check to ensure that the impedance between the electrode and a reference electrode is within a predetermined range. For example, for an EEG, the target impedance may be less than 10 kiloohms with the target being 5 kiloohms. As such, the predetermined range may be from approximately 5 kiloohms to approximately 10 kiloohms. Current techniques often require removal of an electrode in an instance in which the impedance is outside of the predetermined range, in order to perform additional skin preparation and/or abrasive techniques to the skin. Only once the skin is prepared again can the electrode be replaced for use. As such, in an instance in which the impedance is outside of a predetermined range, a large amount of time is required to get the impedance adjusted within the predetermined range. Various embodiments of the present disclosure cure the deficiencies of current techniques by allowing for an area to be abraded by the electrode in an instance in which the impedance is outside of the predetermined range without having to remove the electrode from the skin. Thereby, the amount of time required to perform electrode measurements is reduced without impacting performance.

Various embodiments of the present disclosure include an impedance determination electrode system that includes an abrasion electrode to improve technician workflow and reduce the amount of time required to reduce the skin-to-electrode impedance. Various embodiments include a conductive, abrasion component of the abrasion electrode attached to a battery powered abrasion motor which can automatically abrade the skin (e.g., cause movement of the abrasion electrode). The workflow improvement comes from an integrated impedance determination device that notifies the technician when the current skin location reaches a target impedance. In order to monitor the impedance, two electrodes are required; one for recording (e.g., the abrasion electrode) and one for reference (e.g., the reference electrode). The abrasion electrode attached to the motor may be the recording electrode, and a second sticky hydrogel electrode (e.g., a hydrogel pad) may be used as the reference electrode, although alternative electrodes may suffice. Both the recording and reference electrodes are integrated into a detachable, consumable, single-use element. The battery powered motor and/or the impedance determination device may be part of a device that is reusable.

An example method of using the impedance determination electrode system includes an operator (e.g., an EEG technician) determining electrode locations. The operator then removes the single-use element (e.g., including the abrasion electrode 105 and the reference electrode 115) from package and attaches to reusable, battery powered, handheld motor device (e.g., housing 110). The operator may attach the reference electrode (e.g., a sticky, hydrogel electrode) to a mastoid of patient (e.g., the second skin location). The operator may attach the abrasion electrode to a first skin location and abrade the first skin location by powering on the abrasion motor. The impedance determination device may monitor the impedance between the abrasion electrode 105 and the reference electrode 115. Once the impedance reaches the predetermined range of impedance, an indicator (e.g., a user indication device) on the handheld device may alert the operator. For example, the operator may be notified once the impedance is within the range of 5 kiloohms to 10 kiloohms. Example indicators may be visual (e.g., red light/green light, digital screen, etc.), audible (e.g., audible tone), and/or tactile (e.g., vibration). In various embodiments, the predetermined threshold of impedance may be adjustable (e.g., based on the usage). Additionally or alternatively, once the predetermined range of impedance is achieved, the abrasion motor may automatically stop (e.g., ending the abrading of the skin).

The operations may be repeated for additional skin locations. For example, the abrasion electrode may be replaced with another electrode (e.g., a sticky, hydrogel electrode) at the first skin location once the target impedance range has been achieved and the abrasion electrode may be used at additional skin locations. Alternatively, the abrasion electrode may remain at the first skin location and an additional abrasion electrode may be used at different skin locations. For example, in an instance in which the system is measuring brain wave or EEG signals, electrodes may be placed at one or several locations on the patient's scalp.

In various embodiments, the impedance may be monitored intermittently or continuously. In various embodiments, an exfoliating gel may be applied to the abrasion electrode prior to abrading the skin of the patient. Types of abrasion motors used may include linear motors, ultrasonic motors, rotational motors, and/or the like. In various embodiments, the single-use element (e.g., the abrasion electrode and/or the reference electrode) may be made of soft, bristled material or silicone-like material. In various embodiments, the single-use element may integrate conductive gel or paste to simultaneously abrade and deposit electrolytes into the skin.

### III. With Reference to the Figures

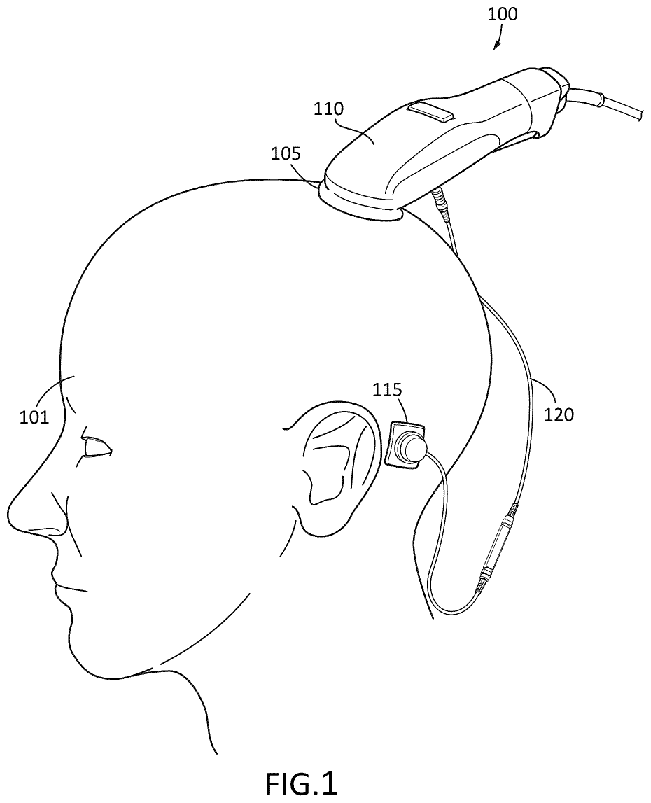

Referring now to FIG. 1, an impedance determination electrode system 100 is shown positioned on the head of a patient 101. In such an example, the impedance determination electrode system 100 may be used for an EEG. While the impedance determination electrode system 100 is shown positioned on the head of a patient 101, the impedance determination electrode system 100 could be used for any operations in which an electrode is used (e.g., other types of testing using electrodes). In various embodiments, the impedance determination electrode system 100 includes an abrasion electrode 105, a housing 110 (e.g., including an abrasion motor and an impedance determination device), a reference electrode 115, and a lead-wire 120 connecting the abrasion electrode 105 and the reference electrode 115. As shown, the abrasion electrode 105 is positioned at a first skin location of the patient 101 and the reference electrode 115 is positioned at a second skin location of the patient 101.

In various embodiments, an impedance determination device (not shown) may be part of the impedance determination system 100. The impedance determination device may transmit, via lead-wire 120, a small test current through the electrode to the skin to measure the voltage drop and resistance, calculating and monitoring the impedance. The impedance determination device may monitor the impedance between the abrasion electrode 105 and the reference electrode 115.

In various embodiments, the impedance determination system 100 may also include a user indication device. The user indication device may be part of or separate from the impedance determination device. The user indication device causes a notification to be provided to a user in an instance in which the impedance between the abrasion electrode and the reference electrode is within a predetermined range of impedance. The impedance determination system 100 may include one or more processing device(s) and memory device(s) to determine the impedance, determine whether the impedance is within a predetermined range, determine the notification to a user, and/or the like.

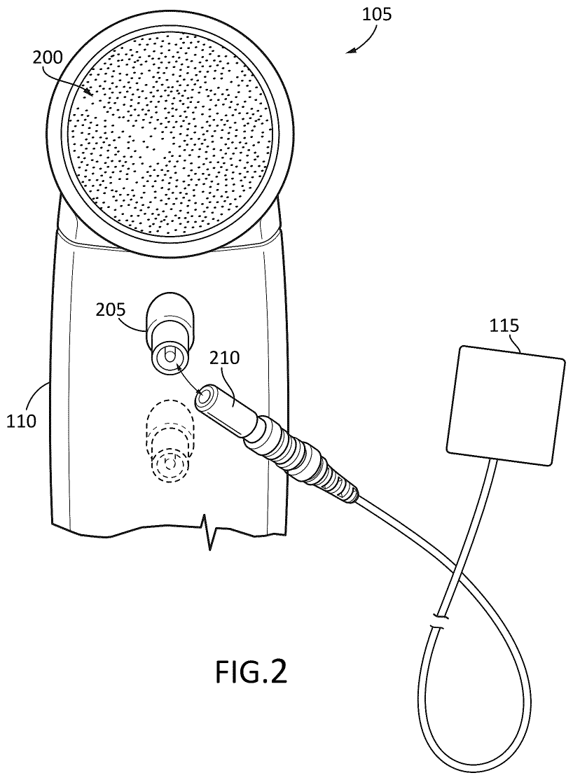

The abrasion electrode 105 may include an abrasion component and an electrode component. The abrasion component abrades the skin of the patient upon actuation by the abrasion motor. The abrasion component may include a means for chemical and/or physical abrasion of the skin. The electrode component electrically engages with the patient (e.g., to perform an EEG) to allow for reliable signal transmission with minimal noise and low impedance. The abrasion component may be part of the electrode component (e.g., part of the single use portion 200 shown in FIG. 2).

In various embodiments, the electrode component may be gelled or dry. Example gelled electrodes include wet gels and hydrogels. A gelled electrolyte provides a conductive medium between the skin and electrode allowing the current to pass from the skin to electrode easier. As the major portion of electrolytes present in tissue fluids and sweat are sodium, potassium, and chloride, the electrolytes used in the electrode component (e.g., the electrode gels) may be sodium chloride and potassium chloride. The electrolytes in the electrode component (e.g., sodium chloride and potassium chloride) provide good electrical conductivity of the gel and increase the conductivity of the skin as the electrolytes diffuse into the skin due to the existing concentration gradient.

In an instance in which the electrode component includes a wet gel electrode, the electrode component may include a gel-impregnated sponge, with adhesive around the outside to ensure hold. Wet gel electrodes have a very high water concentration, actively hydrating the skin and reducing the resistivity in the outer layers. Wet gel electrodes also typically have a higher concentration of electrolytes than hydrogels, which produces a much lower impedance. In various embodiments, the electrode component may include a hydrogel. A hydrogel provides a less harsh interaction with the skin, allowing for longer testing periods.

In various embodiments, the electrode component may include a dry electrode. A dry electrode may be a contact or non-contact electrode. Non-contact dry electrodes are capacitively coupled to the skin, hence, the non-contact dry electrodes give a very small signal amplitude and are highly sensitive to motion artifacts since motion will change the skin-electrode capacity. Direct contact dry electrodes may be either electrodes with microscale needles on the surface that penetrate the stratum corneum and/or electrodes that are in direct contact with the skin but without penetration. Since microscale needles are conductive (conductive bulk material or coating), the direct contact dry electrode may lower the overall electrode-skin impedance. Direct contact electrodes may be equipped with macroscale pins to improve functionality on hairy skin, since the hair can be positioned in the space in between the pins and hence an improved electrode-skin contact results.

In various embodiments, the abrasion electrode may also have an abrasion component. The abrasion component may be part of the electrode component (e.g., part of the single use portion 200 shown in FIG. 2) and/or distinct from the electrode component (e.g., the electrode component may be adjacent to the abrasion component). The abrasion component may provide an abrasion to the skin of a patient. The abrasion component may have any number of different type of abrasion, such as using an abrading material. In various embodiments, the electrode component may be made of an abrading material. In such an instance, the electrode component may be the same portion of the abrasion electrode as the abrasion component. For example, the single use portion 200 (shown in FIG. 2) may include an electrode that has abrading characteristics. In an instance in which the abrasion motor (discussed herein) is actuated, the abrasion component may abrade the skin of the patient. For example, the electrode may include an abrading material that upon actuation of the abrasion motor may abrade the skin, but upon deactivation of the abrasion motor remains in place and can be used as an electrode.

In various embodiments, the reference electrode 115 may be an electrode that requires manual abrasion and placement by an operator. As such, the reference electrode 115 may be a sticky, hydrogel electrode (e.g., a hydrogel pad). In an example in which the impedance determination electrode system 100 is used for an EEG, the reference electrode 115 may be positioned at the mastoid of the patient. The impedance of the reference electrode 115 may be determined upon placement (e.g., to determine whether the reference electrode 115 is correctly attached). For example, the reference electrode 115 may be tested using traditional electrode procedures. The reference electrode may be removed and/or replaced based on the impedance reading. The placement of the reference electrode 115 is the only electrode that requires manual abrasion. As such, the system herein allows for multiple electrodes (e.g., multiple abrasion electrodes) to be positioned while only requiring manual abrasion of the reference electrode.

In an instance in which the impedance determination electrode system 100 is used to collect EEG or brain wave signals, common locations for the electrodes include frontal, parietal, mastoid, central, and/or occipital. For example, in an instance in which the impedance determination electrode system 100 is used to collect EEG or brain wave data, at least one reference electrode 115 may be placed on the occipital (e.g., the second skin location) and referenced against the abrasion electrode 105 placed on the mastoid (e.g. the first skin location). In various embodiments, additional reference electrodes 115 placed to obtain reference readings (e.g., a second reference electrode may be positioned on the patient to provide a second channel of data from the central location). Any number of abrasion electrode(s) 105 and/or reference electrode(s) 115 may be used for a patient.

In various embodiments, the housing 110 may be a reusable, handheld device that is connected to the abrasion electrode and the reference electrode 115. The housing 110 may house the abrasion motor, the impedance determination device, and/or the user indication device. In various embodiments, each of the abrasion motor, the impedance determination device, and/or the user indication device may share components. The abrasion motor may be connected to the abrasion electrode in order to create movement of the abrasion electrode to abrade the skin. The impedance determination device may monitor and/or otherwise read the impedance between the abrasion electrode and the reference electrode 115. The user indication device may cause a notification to be provided to a user in an instance in which the impedance between the abrasion electrode and the reference electrode is within a predetermined range of impedance.

While the housing 110 is described as a handheld device herein, various embodiments may contemplate different housings for the housing 110 (e.g., a non-handheld station may be provided). Additionally, while the abrasion motor, the impedance determination device, and the user indication device are shown in the housing, various embodiments may include one or more of the abrasion motor, the impedance determination device, or the user indication device in different housings (e.g., a housing for the abrasion motor and a housing for the impedance determination device and the user indication device).

The housing 110 may be detachable from the rest of the abrasion electrode (e.g., detachable from the single use portion 200). As such, the abrasion motor and other components within the housing may be used across multiple abrasion electrodes. For example, the housing 110 may be used across multiple abrasion electrodes to correctly attach the electrode within the predetermined impedance. In various embodiments, the housing 110 may be detached before actual use of the abrasion electrode (e.g., the single use portion 200 may be detached from the housing 110 before the

[symbol]

Referring now to FIG. 2, an illustration of an example abrasion electrode 105 and a reference electrode 115 of the impedance determination electrode system is provided. The abrasion electrode 105 may include a housing 110 and a single use portion 200. The housing 110 may be a handheld device. In various embodiments, the housing 110 may contain or otherwise be connected to the abrasion motor discussed herein. The housing 110 may be connected to the single use portion 200. For example, as shown in FIG. 2, the single use portion 200 may be received by the housing 110 (e.g., connected to the abrasion motor). The abrasion electrode 105 may include an electrode component and an abrasion component. The electrode component and/or the abrasion component may be part of the single use portion 200. In various embodiments, the abrasion component may be the same portion of the abrasion electrode as the electrode component. For example, the abrasion component may be made out of a conductive material, such that the abrasion component acts as an electrode.

In various embodiments, the abrasion component may be made out of an abrasion material. In various embodiments, an abrasion gel (e.g., a hydrogel) may be applied to the abrasion component before being applied to the skin of the patient. For example, the abrasion gel may be applied to the abrasion component and upon actuation of the abrasion motor (upon placement of the abrasion electrode).

In various embodiments, the lead-wire 120 may be connected to the abrasion electrode 105 via the housing 110. As shown, the housing 110 may include a connector 205 that receives a connector 210 of the lead-wire 120. In such an embodiment, the connector 205 may electrically connect the reference electrode 115 to the abrasion electrode 105. In various embodiments, the lead-wire 120 may be permanently connected to the housing 110 (e.g., the lead-wire may detach from a reference electrode and attach to another reference electrode).

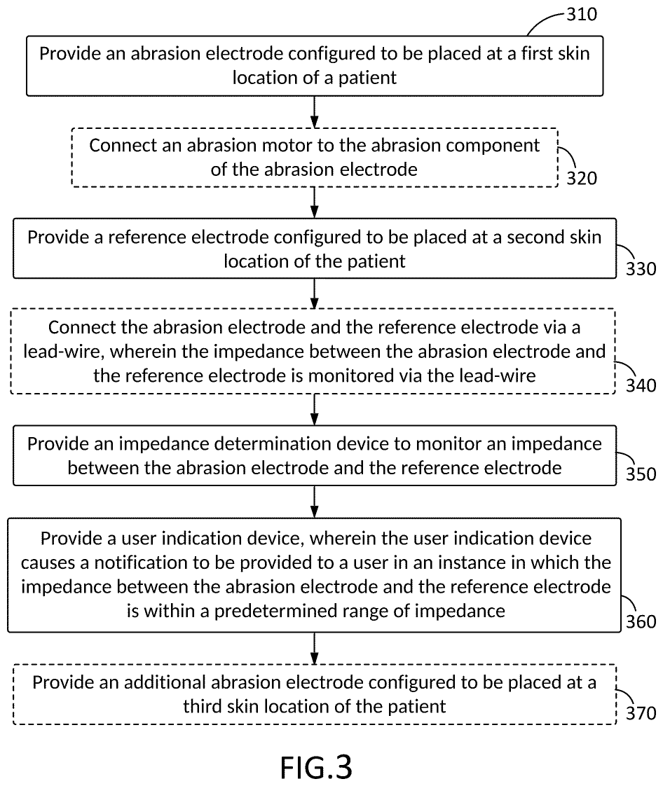

Referring now to FIG. 3, a flowchart of an example method of manufacturing an impedance determination electrode system. The impedance determination electrode system 100 may be the impedance determination electrode system of FIGS. 1 and 2, and/or any other embodiments herein.

Referring now to Block 310 of FIG. 3, the method includes providing an abrasion electrode configured to be placed at a first skin location of a patient, the abrasion electrode comprises an abrasion component to abrade the first skin location of the patient and an electrode component to electrically engage with the patient. The abrasion electrode 105 is capable of automatically abrading the skin (e.g., via the abrasion component) based on the operations herein. The abrasion can be completed after the abrasion electrode is positioned at a first skin location, thereby allowing for faster implementation.

The abrasion electrode may include an electrode component that is a single use element (e.g., part of the single use portion 200). The electrode component of the abrasion electrode 105 may be made of soft, bristled material, silicone-like material, and/or the like. The abrasion electrode may be connected to the reference electrode 115 via a lead wire. As such, the electrode component of the abrasion electrode 105 and the reference electrode 115 may be packaged or otherwise provided together as a single use element. Alternatively, the reference electrode 115 may be used across multiple abrasion electrodes, such that the lead wire(s) may be detachable to allow other abrasion electrodes to connect to the reference electrode 115. In various embodiments, the lead wire may be detachable from the abrasion electrode (e.g., to attach another abrasion electrode to the reference electrode). Additionally or alternatively, the lead wire may be detached from the reference electrode and a new lead wire connected to the additional abrasion electrode may be attached to the reference electrode.

In various embodiments, the abrasion motor and/or the abrasion component of the abrasion electrode 105 may be reusable. In various embodiments, at least a portion of the abrasion component may also be part of the single use element (e.g., single use portion 200 of FIG. 2) that includes the electrode component of the abrasion electrode 105. For example, the single use portion 200 shown in FIG. 2 may include both the abrasion component and the electrode component and the abrasion component may be detachable from the rest of the abrasion electrode (e.g., replaced with single use element).

Referring now to optional Block 320 of FIG. 3, the method includes connecting an abrasion motor to the abrasion component of the abrasion electrode. The abrasion motor may provide movement of the abrasion component to abrade.

The abrasion motor may be part of the housing 110 of the abrasion electrode 105. The abrasion motor may be housed within a handheld device (e.g., the housing 110 is shown in FIG. 2 as a handheld device and the abrasion motor may be housed within the handheld device or be connected to the handheld device). The abrasion motor may be a linear motor, ultrasonic motor, rotational motor, and/or the like. The abrasion motor may provide movement of the abrasion component to abrade the skin while the abrasion electrode 105 is positioned at the first skin location.

As shown in FIG. 2, the single use portion 200 may be detachable from the housing 110. In various embodiments, the single use portion 200 may snap or otherwise fit into connection with the housing 110, creating an electrical and/or mechanical connection between the abrasion motor and the single use portion 200. The connection of the single use portion 200 and the housing 110 may also provide electrical connection with the impedance determination device and/or the user indication device. The housing 110 may include a button to detach a single use portion 200 (e.g., upon placement of the abrasion electrode on the skin of the patient). As such, the housing 110 may be used across multiple abrasion electrodes (e.g., the housing 110 may be used to place multiple abrasion electrodes at various locations on the skin of a patient).

As discussed herein, the abrasion motor speed and/or power may be adjusted based on the impedance readings between the abrasion electrode 105 and the reference electrode 115. The abrasion motor may be turned off or have the power reduced in an instance in which the impedance between the abrasion electrode 105 and the reference electrode 115 reaches the predetermined impedance range between the abrasion electrode 105 and the reference electrode 115. The power provided to the abrasion motor may be increased in an instance in which the impedance is not within the predetermined impedance range between the abrasion electrode 105 and the reference electrode 115.

Referring now to Block 330 of FIG. 3, the method includes providing a reference electrode configured to be placed at a second skin location of the patient. In various embodiments, the reference electrode may be any number of different kinds of electrodes. For example, the reference electrode may include a hydrogel pad. The reference electrode may be positioned using a typical abrasion process (e.g., using hydrogel and/or a blunt object). For example, the reference electrode may be a sticky, hydrogel electrode that is positioned at a second skin location (e.g., mastoid of the patient).

The reference electrode 115 may be a single use element. For example, the single use reference electrode may be packaged with the single use portion 200 of the abrasion electrode via a wire. In various embodiments, a kit of single use elements may include at least one abrasion electrode (e.g., the single use portion 200 of the abrasion electrode), a reference electrode, and at least one lead-wire 120. In such an embodiment, reference electrode may be connected to each of the abrasion electrodes (e.g., individually one after another using a single lead-wire or simultaneously using multiple lead-wires).

Referring now to optional Block 340 of FIG. 3, the method includes connecting the abrasion electrode and the reference electrode via a lead-wire. In various embodiments, the impedance between the abrasion electrode and the reference electrode may be monitored via the lead-wire. As discussed herein, the abrasion electrode and the reference electrode may be produced as a single use element with a lead-wire connection.

In various embodiments, the abrasion electrode and the reference electrode may be connected to the impedance determination device via the lead-wire 120. For example, the impedance determination device may be connected to the lead-wire 120 to monitor the impedance between the abrasion electrode and the reference electrode. In various embodiments, the impedance determination device may be connected via lead-wires to multiple abrasion electrodes and the reference electrode (e.g., to measure the impedance between the reference electrode and each of the abrasion electrode(s)).

Referring now to Block 350 of FIG. 3, the method includes providing an impedance determination device to monitor an impedance between the abrasion electrode and the reference electrode. The monitoring of the impedance between the abrasion electrode and the reference electrode may be continuous or intermittent. The impedance may be monitored via the lead-wire connecting the abrasion electrode and the reference electrode.

In various embodiments, the impedance determination device monitors the impedance between the abrasion electrode and the reference electrode during placement of the abrasion electrode. Placement may include any portion of time in which the abrasion electrode is applied, but not quite within the predetermined range of impedance. For example, the impedance may be monitored while the abrasion electrode is being placed at the first skin location and continued until at least when the impedance is within the predetermined range of impedance.

The first skin location may be abraded via the abrasion electrode 105 and the impedance between the abrasion electrode and the reference electrode may be monitored during the abrasion and/or after the abrasion. For example, upon placement of the abrasion electrode at the first skin location and the reference electrode at the second skin location, the impedance determination device may monitor or record the impedance between the abrasion electrode and the reference electrode before abrasion begins and then again after the abrasion is conducted.

In various embodiments, the first skin location may be abraded one or more times, based on the impedance between the abrasion electrode and the reference electrode. The abrasion may be iterative (e.g., the abrasion electrode 105 may abrade the skin, the impedance determination device may determine the impedance between the abrasion electrode and the reference electrode, and then the abrasion electrode may abrade the first skin location again in an instance in which the impedance between the abrasion electrode and the reference electrode is not within the predetermined range). As such, the number of abrading and/or the amount of abrasion may be based on the impedance between the abrasion electrode and the reference electrode.

In various embodiments, the impedance between the abrasion electrode and the reference electrode may determine the amount of abrasion and the duration of the abrasion to be completed on the first skin location. In various embodiments, the abrasion motor may increase or decrease the movement of the abrasion electrode 105 based on the difference between the measured impedance between the abrasion electrode and the reference electrode, and the predetermined impedance range. For example, less abrasion may be need in an instance in which the measured impedance is close to the predetermined impedance range. Additionally or alternatively, the length of the abrasion may be adjusted based on the measured impedance between the abrasion electrode and the reference electrode. For example, the closer the measure impedance is to the predetermined impedance range, the less abrasion time that is need.

In various embodiments, the monitoring of the impedance between the abrasion electrode and the reference electrode may be continued once the predetermined range of impedance has been met (e.g., to ensure that the abrasion electrode is still applied to the skin correctly). Alternatively, the monitoring of the impedance between the abrasion electrode and the reference electrode may be ceased once the predetermined range of impedance has been met. The monitoring of the impedance may be related to the motor

[symbol]

Referring now to Block 360 of FIG. 3, the method includes providing a user indication device. The user indication device causes a notification to be provided to a user in an instance in which the impedance between the abrasion electrode and the reference electrode is within a predetermined range of impedance. The predetermined range of impedance may be based on the data being monitored by the electrode. For example, the amount of impedance may be minimized in order to get useful readings. Example indicators from the user indication device may be visual (e.g., red light/green light, digital screen, etc.), audible (e.g., audible tone), and/or tactile (e.g., vibration). In various embodiments, the user indication device may be part of one or more other components herein. For example, the user indication device and the impedance determination device may be a singular device. Alternatively, the user indication device and the impedance determination device may be in communication with one another (e.g., such that the user indication device can cause the notification to be provided once the impedance is within the predetermined range of impedance).

In various embodiments, the desired impedance between the impedance between the abrasion electrode and the reference electrode may be as close to zero as possible. For example, an impedance of zero indicates that the abrasion electrode is affixed to the skin with the same impedance between electrode and skin as the reference electrode. As such, the predetermined range of impedance may be a small value that may also indicate a good connection between the abrasion electrode and the skin. In various embodiments, the predetermined range of impedance between the abrasion electrode and the reference electrode may be between approximately 0 kiloohms and approximately 10 kiloohms. In various embodiments, the predetermined range of impedance between the abrasion electrode and the reference electrode may be between approximately 5 kiloohms and approximately 10 kiloohms. In various embodiments, the predetermined range of impedance between the abrasion electrode and the reference electrode may be adjustable. For example, the predetermined range of impedance between the abrasion electrode and the reference electrode

[symbol]

Referring now to optional Block 370 of FIG. 3, the method includes providing an additional abrasion electrode configured to be placed at a third skin location of the patient. In various embodiments, any number of abrasion electrodes may be used (e.g., as each of the electrodes used during an EEG). As such, any number of abrasion electrodes may be provided. Such additional abrasion electrodes may be connected to the same abrasion motor and/or additional abrasion motors may be provided. For example, the additional abrasion electrode may be another of the single use portion 200 shown in FIG. 2 and the housing 110 may be detached from the abrasion electrode and then attached to the additional electrode.

In various embodiments, each of the additional abrasion electrodes (e.g., single use portion 200) include an abrasion component to abrade a skin location (e.g., the third skin location, a fourth skin location, a fifth skin location, etc.) and an electrode component to electrically engage with the patient. The impedance determination device may monitor an impedance between each additional abrasion electrode and the reference electrode. In various embodiments, additional reference electrode(s) may also be provided (e.g., one or more electrodes may be connected to a first reference electrode and one or more electrodes may be connected to a second reference electrode).

In various embodiments, the same housing 110 (e.g., handheld device shown in FIG. 2) with a new single use element may be used to apply the additional abrasion electrodes. For example, the housing 110 may detach from the single use portion 200 (e.g., the electrode component and/or the abrasion component) and attach to a new single use element that may then be placed as an additional abrasion electrode. As such, the electrode component of the abrasion electrode may be considered the abrasion electrode upon placement.

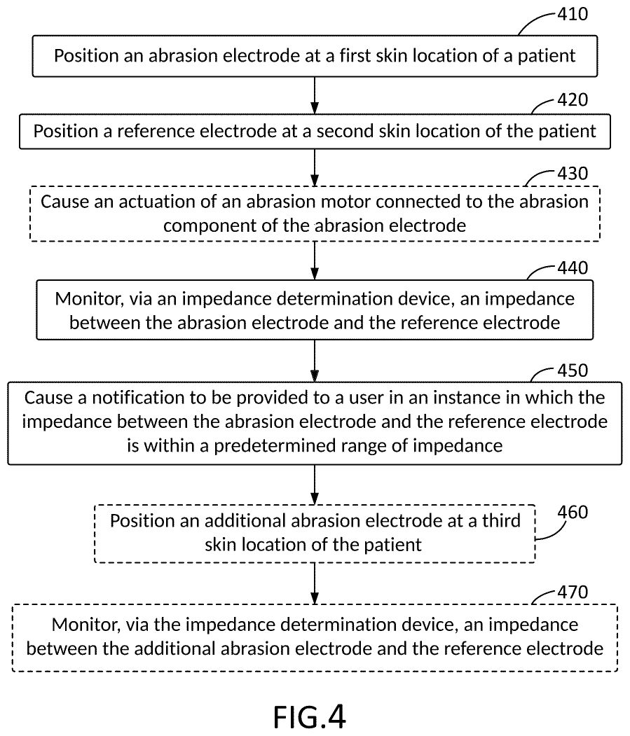

Referring now to FIG. 4, a flowchart of an example method of using an impedance determination electrode system is provided. The method of FIG. 4 may be carried out by any embodiments of the impedance determination electrode system discussed herein. For example, the method may be carried out by the system discussed in reference to FIGS. 1 and 2, as well as the system discussed in reference to a method of manufacturing in FIG. 3.

Referring now to Block 410 of FIG. 4, the method includes positioning an abrasion electrode at a first skin location of a patient. The abrasion electrode 105 includes an abrasion component to abrade the first skin location of the patient and an electrode component to electrically engage with the patient. The abrasion electrode may be any of the abrasion electrodes discussed herein. As discussed herein, the abrasion electrode 105 may include a single use portion 200 that defines an electrode portion and, in some instances, an abrasion portion. The single user portion 200 may have an abrading material that is actuated via the abrasion motor.

Referring now to Block 420 of FIG. 4, the method includes positioning a reference electrode at a second skin location of the patient. The reference electrode 115 may be attached to the second skin location via a manual abrasion and attachment. The reference electrode 115 may be any number of different type electrodes. For example, the reference electrode 115 may include a hydrogel pad.

The second skin location may be any number of different locations on a patient. In an instance in which the system is used for an EEG, the second skin location may be the mastoid of the patient. In various embodiments, the second skin location may be any location in which an abrasion electrode is not to be applied.

Referring now to optional Block 430 of FIG. 4, the method includes causing an actuation of an abrasion motor connected to the abrasion component of the abrasion electrode. In various embodiments, the actuation of the abrasion motor provides movement of the abrasion component to abrade. The abrasion motor causes the abrasion component of the abrasion electrode 105 to move and contact the skin at the first skin location.

As discussed above in reference to FIG. 1, the abrasion motor may be within a housing 110. The abrasion motor may be automatically actuated (e.g., in an instance in which the impedance between the abrasion electrode and the reference electrode is outside of the predetermined range of impedance) and/or manually actuated (e.g., an operator may turn on the abrasion motor).

Referring now to Block 440 of FIG. 4, the method includes monitoring, via an impedance determination device, an impedance between the abrasion electrode and the reference electrode. The impedance may be monitored as discussed above in reference to Block 350 of FIG. 3. As such, the impedance between the abrasion electrode and the reference electrode may be monitored during the placement of the abrasion electrode.

Referring now to Block 450 of FIG. 4, the method includes causing a notification to be provided to a user in an instance in which the impedance between the abrasion electrode and the reference electrode is within a predetermined range of impedance. The notification may be provided by the user indication device as discussed above in reference to Block 360 of FIG. 3.

Referring now to optional Block 460 of FIG. 4, the method includes positioning an additional abrasion electrode at a third skin location of the patient. The additional abrasion electrode comprises an abrasion component to abrade the third skin location of the patient and an electrode component to electrically engage with the patient. Referring to FIG. 2, the single use portion 200 of the abrasion electrode may be detached from the housing 110 and another single use portion 200 (e.g., an additional abrasion electrode) may be attached to the housing 110. The additional abrasion electrode may be electrically connected to the reference electrode 115 via the lead-wire 120 (e.g., that is attached to the housing 110 in FIG. 2). The operations of FIG. 4 may be repeated on any number of skin locations using the same or different abrasion electrodes. As such, the operations herein may be repeated for additional skin location.

In various embodiments, the same reference electrode 115 may be used for the additional abrasion electrodes. For example, a single reference electrode 115 may be used for any number of abrasion electrodes discussed herein. In various embodiments, additional reference electrodes may also be used. For example, the housing 110 may be used to apply abrasion electrodes on the skin of different patients and each patient may have a reference electrode. Alternatively, a single patient may have multiple reference electrodes (e.g., to provide additional certainty of testing results).

Referring now to optional Block 470 of FIG. 4, the method includes monitoring, via the impedance determination device, an impedance between the additional abrasion electrode and the reference electrode. The impedance determination device may be used across multiple electrodes at various skin locations. The impedance may be monitored across each abrasion electrode (e.g., abrasion electrode and/or any additional abrasion electrodes) simultaneously and/or sequentially (e.g., a first abrasion electrode may be positioned, then a second abrasion electrode may be positioned, etc.).

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, the following is claimed:

1. An electrode system for determining impedance, the system comprising:
an abrasion electrode configured to be placed at a first skin location of a patient, the abrasion electrode comprises an abrasion component to abrade the first skin location of the patient and an electrode component to electrically engage with the patient;
a reference electrode configured to be placed at a second skin location of the patient;
an impedance determination device, wherein the impedance determination device monitors an impedance between the abrasion electrode and the reference electrode; and
a user indication device, wherein the user indication device causes a notification to be provided to a user in an instance in which the impedance between the abrasion electrode and the reference electrode is within a predetermined range of impedance.

2. The electrode system of claim 1, further comprising an abrasion motor connected to the abrasion component of the abrasion electrode, wherein the abrasion motor provides movement of the abrasion component to abrade.

3. The electrode system of claim 1, wherein the predetermined range of impedance is between 5 kiloohms and 10 kiloohms.

4. The electrode system of claim 1, wherein the impedance determination device monitors the impedance between the abrasion electrode and the reference electrode during placement of the abrasion electrode.

5. The electrode system of claim 1, wherein the abrasion electrode and the reference electrode are connected via a lead-wire, wherein the impedance between the abrasion electrode and the reference electrode is monitored via the lead-wire.

6. The electrode system of claim 1, wherein the reference electrode comprises a hydrogel pad.

7. The electrode system of claim 1, further comprising an additional abrasion electrode configured to be placed at a third skin location of the patient, wherein the additional abrasion electrode comprises an abrasion component to abrade the third skin location of the patient and an electrode component to electrically engage with the patient, wherein the impedance determination device monitors an impedance between the additional abrasion electrode and the reference electrode.

8. A method of using an impedance determination electrode system, the method comprising:
providing an abrasion electrode configured to be placed at a first skin location of a patient, the abrasion electrode comprises an abrasion component to abrade the first skin location of the patient and an electrode component to electrically engage with the patient;
providing a reference electrode configured to be placed at a second skin location of the patient;
providing an impedance determination device to monitor an impedance between the abrasion electrode and the reference electrode; and
providing a user indication device, wherein the user indication device causes a notification to be provided to a user in an instance in which the impedance between the abrasion electrode and the reference electrode is within a predetermined range of impedance.

9. The method of claim 8, further comprising connecting an abrasion motor to the abrasion component of the abrasion electrode, wherein the abrasion motor provides movement of the abrasion component to abrade.

10. The method of claim 8, wherein the predetermined range of impedance is between 5 kiloohms and 10 kiloohms.

11. The method of claim 8, wherein the impedance determination device monitors the impedance between the abrasion electrode and the reference electrode during placement of the abrasion electrode.

12. The method of claim 8, further comprising connecting the abrasion electrode and the reference electrode via a lead-wire, wherein the impedance between the abrasion electrode and the reference electrode is monitored via the lead-wire.

13. The method of claim 8, wherein the reference electrode comprises a hydrogel pad.

14. The method of claim 8, further comprising providing an additional abrasion electrode configured to be placed at a third skin location of the patient, wherein the additional abrasion electrode comprises an abrasion component to abrade the third skin location of the patient and an electrode component to electrically engage with the patient, wherein the impedance determination device monitors an impedance between the additional abrasion electrode and the reference electrode.

15. A method of using an impedance determination electrode system, the method comprising:
positioning an abrasion electrode at a first skin location of a patient, the abrasion electrode comprises an abrasion component to abrade the first skin location of the patient and an electrode component to electrically engage with the patient;
positioning a reference electrode at a second skin location of the patient;
monitoring, via an impedance determination device, an impedance between the abrasion electrode and the reference electrode; and
causing a notification to be provided to a user in an instance in which the impedance between the abrasion electrode and the reference electrode is within a predetermined range of impedance.

16. The method of claim 15, further comprising causing an actuation of an abrasion motor connected to the abrasion component of the abrasion electrode, wherein the abrasion motor provides movement of the abrasion component to abrade.

17. The method of claim 15, wherein the predetermined range of impedance is between 5 kiloohms and 10 kiloohms.

18. The method of claim 15, wherein the impedance between the abrasion electrode and the reference electrode is monitored during placement of the abrasion electrode.

19. The method of claim 15, wherein the reference electrode comprises a hydrogel pad.

20. The method of claim 15, further comprising:
positioning an additional abrasion electrode at a third skin location of the patient, wherein the additional abrasion electrode comprises an abrasion component to abrade the third skin location of the patient and an electrode component to electrically engage with the patient; and
monitoring, via the impedance determination device, an impedance between the additional abrasion electrode and the reference electrode.
